(19) United States
(12) Patent Application Publication
(10) Pub. No.: US 2021/0196319 A1
(43) Pub. Date: Jul. 1, 2021

(54) TROCAR WITH INTERNAL CAMERA PROVIDING TILTED VIEW ANGLE

(71) Applicant: BIOSENSE WEBSTER (ISRAEL) LTD, Yokneam (IL)

(72) Inventors: Yehuda Algawi, Binyamina (IL); Assaf Govari, Haifa (IL); Ilya Sitnitsky, Nahariya (IL); Andres Claudio Altmann, Haifa (IL); Gili Attias, Haifa (IL)

(21) Appl. No.: 16/729,434

(22) Filed: Dec. 29, 2019

(51) Int. Cl. A61B 17/34; A61B 1/313

(52) U.S. Cl. CPC A61B 17/3476; A61B 1/3132; A61B 17/3403

(57) ABSTRACT

A trocar for insertion into an organ of a patient includes a cannula having a distal opening, a channel inside the cannula, and an optical assembly including a camera. The optical assembly is disposed at a distal end of the channel and is configured to provide camera images of the distal opening with a field-of-view (FOV) that is tilted relative to a longitudinal axis of the cannula.

### FIELD OF THE INVENTION

The present invention relates generally to invasive medical tools, and particularly to invasive medical tools incorporating a camera.

### BACKGROUND OF THE INVENTION

Techniques for image-guided probing of an organ of a patient were previously proposed in the patent literature. For example, U.S. Patent Application Publication 2015/0272694 describes a surgical device includes a plurality of cameras integrated therein. The view of each of the plurality of cameras can be integrated together to provide a composite image. A surgical tool that includes an integrated camera may be used in conjunction with the surgical device. The image produced by the camera integrated with the surgical tool may be associated with the composite image generated by the plurality of cameras integrated in the surgical device. The position and orientation of the cameras and/or the surgical tool can be tracked, and the surgical tool can be rendered as transparent on the composite image.

As another example, U.S. Pat. No. 5,188,093 describes a hand-held portable arthroscope that has a camera assembly and a disposable scope assembly rotatably attached to the distal end of the camera assembly. A probe which includes a fiber optic image guide and a plurality of optical illuminating fibers is rotatably mounted on the scope assembly, and is insertable into a body for imaging the internal structure of the body. The illuminating fibers extend through the scope assembly and are connected in light communication with a quartz halogen lamp for illuminating the internal body structure being imaged. To gather light from the illuminated internal structure of a body into which the probe has been inserted, a GRIN rod is attached to the distal end of the image guide. The image guide is bent near its distal end to offset the axis of the GRIN rod from the axis of the camera assembly, to enhance the field of view of the arthroscope when the scope assembly, which supports the image guide with GRIN rod, is rotated. Focusing optics are mounted in the camera assembly in light communication with the image guide. These focusing optics are axially movable within the camera assembly for focusing the image from the image guide. Light which passes through the focusing optics enters a camera head which is also mounted in the camera assembly.

U.S. Patent Application Publication 2006/0232664 describes a videoendoscopic surgery training system that includes a housing defining a practice volume in which a simulated anatomical structure is disposed. Surgical instruments can be inserted into the practice volume to access the anatomical structure. A digital video camera is disposed within the housing to image the anatomical structure on a display. The position of the digital video camera is supported within the practice volume by a camera bracket that enables a position of the video camera relative to the bracket to be selectively changed, thereby changing a viewing angle achieved by the video camera. In one embodiment the camera bracket is coupled to a boom, a proximal end of which extends outside the housing to enable additional positioning of the digital video camera by user adjustment of the proximal end of the boom.

### SUMMARY OF THE INVENTION

An embodiment of the present invention provides a trocar for insertion into an organ of a patient, the trocar including a cannula having a distal opening, a channel inside the cannula, and an optical assembly including a camera. The optical assembly is disposed at a distal end of the channel and is configured to provide camera images of the distal opening with a field-of-view (FOV) that is tilted relative to a longitudinal axis of the cannula.

In some embodiments, the optical assembly is configured to modify a tilt angle of the FOV in response to an adjustment by a user.

In some embodiments, the optical assembly includes a tiltable mirror that is configure to modify the tilt angle of the FOV by deflect a direction of view of the camera.

In an embodiment, the tiltable mirror is a MEMS mirror.

In another embodiment, the optical assembly includes a tiltable element on which the camera is mounted so as to deflect a direction of view of the camera. In yet another embodiment, the tiltable element is a MEMS element.

There is additionally provided, in accordance with another embodiment of the present invention, a method, including inserting a trocar into an organ of a patient, the trocar including a cannula having a distal opening, and a channel inside the cannula. Using an optical assembly including a camera that is disposed at a distal end of the channel, camera images are acquired of the distal opening with a field-of-view (FOV) that is tilted relative to a longitudinal axis of the cannula.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a brain procedure using a surgical apparatus comprising a trocar comprising a camera and a tiltable mirror, in accordance with an embodiment of the present invention;

FIG. 2 is a schematic, pictorial illustration of the trocar applied in the brain procedure of FIG. 1, in accordance with an embodiment of the present invention; and

FIG. 3 is a flow chart that schematically illustrates a method and algorithm for acquiring a visual image from the camera of the trocar of FIG. 2, in accordance with an embodiment of the present invention.

### DETAILED DESCRIPTION OF EMBODIMENTS

### Overview

Some invasive medical procedures require a way to visually guide a medical probe to an organ, such as a brain, of a patient. In some invasive procedures, to insert a medical probe or other tool into the body of a patient, a trocar, which serves as a penetrating portal, is first placed in an entry location. In addition to being a portal for the probe, the trocar, which comprises a cannula, may be used for irrigation and to drain bodily fluids, as well as other fluids. Moreover, the trocar may be equipped with a camera to assist in the visual navigation of the probe to target tissue.

For example, brain procedures may require navigating a distal end of a probe inserted into the brain via a hole made in the skull. For the procedure, a trocar with a camera may be inserted to enable a physician to acquire images of a target brain tissue, and a treating probe to be advanced via the trocar and visually guided to treat the target brain tissue, for example, an infected or bleeding brain tissue.

However, the camera has a relatively narrow field of view, and an acquired image may be off-direction, e.g. showing a distal edge of the medical probe, or an image obscured by blood or other matter.

Embodiments of the present invention that are described hereinafter provide a trocar that has, fitted internally to a wall of the cannula, an optical assembly comprising a camera. The assembly is configured to provide camera images of a distal opening of the cannula with a field-of-view (FOV) that is tilted relative to the longitudinal axis of the cannula. The tilt angle aims to provide a clear view, such as of target tissue and/or of a treating probe.

In some embodiments the camera FOV is tilted at a fixed angle relative to the longitudinal axis of the cannula, e.g., at a tilt angle of 10° or other suitable value. In other embodiments, the tilt angle of the camera FOV is adjustable over a range of directions that includes a direction to a center of the distal opening of the cannula. To this end, in an embodiment, a tiltable mirror on the optical path of the camera deflects the direction of view by tilting into a target angle. The mirror may be a MEMS mirror or another type of moving mirror.

However, other solutions may be used to adjust a view of the camera, including other types of moving optics, electro-optic means that can deflect a direction by refraction or diffraction, or by tilting the camera itself using a tiltable camera mount (and thereby avoiding a need for additional elements or functions, such as a tiltable mirror or other types of movable optics, or electro-optic effects).

By optimizing visual image acquisition using a tilted, and in some embodiments direction-adjustable (i.e., tiltable) view of a camera of a trocar, the disclosed technique may improve the outcome of minimally invasive medical procedures.

### System Description

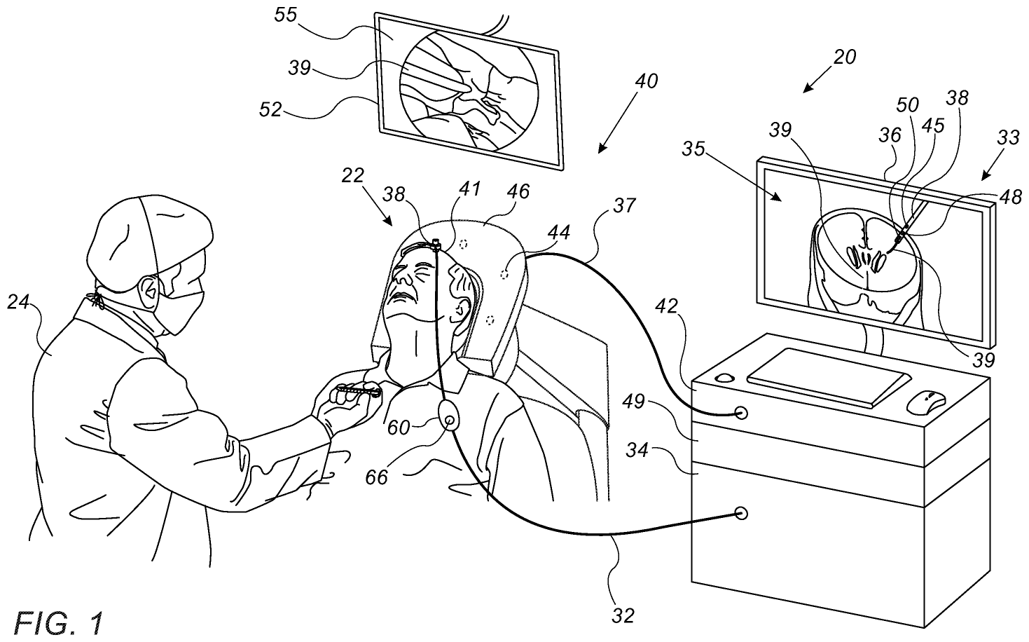

FIG. 1 is a schematic, pictorial illustration of a brain procedure using a surgical apparatus 28 comprising a trocar comprising a camera 48 and a tiltable mirror 50, in accordance with an embodiment of the present invention. Trocar 38 described herein is an example of a trocar having a camera with a tiltable field-of-view (FOV). Other tilting mechanisms other than a tiltable mirror are addressed further below. It is noted that the disclosed techniques are not limited to adjustable-tilt FOV—Alternative embodiments provide trocars in which the camera FOV is tilted at a fixed angle.

In some embodiments, a brain diagnostics and treatment system 20, which comprises surgical apparatus 28, is configured to carry out a brain procedure, such as treating an infection of brain tissue of a patient 22. In the shown embodiment, trocar 38 is used to penetrate the skull so that a physician 24 can insert a probe 39 into a head 41 of patient 22 (insertion not shown) to access brain tissue. Subsequently, probe 39 may be operated using the trocar-attached camera 48.

In the shown embodiment, a cable 32 enters a proximal end of trocar 38 and is electrically wired on its distal end to camera 48 and tiltable mirror 50. A control handle 60 enables the physician to adjust a viewing angle of camera 48 by adjusting a tilt angle of mirror 50. The physician can make the adjustment using a control knob 66.

Control handle 60 may further include additional control elements to assist physician 24 to perform the procedure, such as command buttons to capture an image from camera 48 and to register the image with a reference medical image.

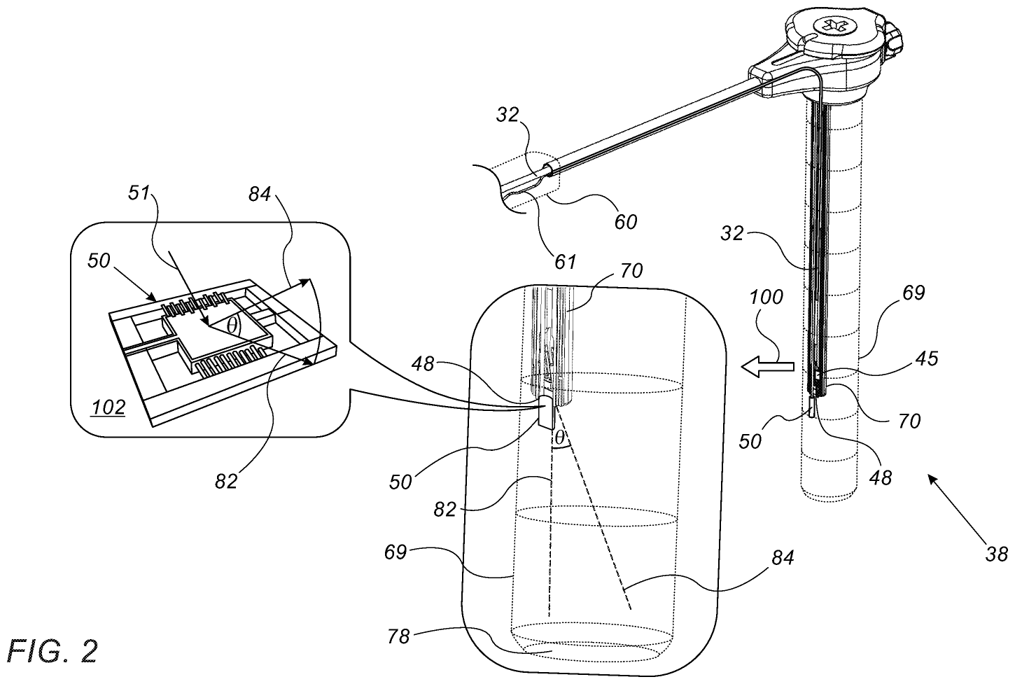

System 20 comprises a magnetic position-tracking system which is configured to track a position of sensor 45 in the brain. The magnetic position-tracking system comprises a location pad 40, which comprises field generators 44 fixed on a frame 46. In the exemplary configuration shown in FIG. 1, pad 40 comprises five field generators 44, but may alternatively comprise any other suitable number of generators 44. Pad 40 further comprises a pillow (not shown) placed under head 41 of patient 22, such that generators 44 are located at fixed, known positions external to head 41. A position sensor 45 that is fitted on a wall of a cannula of trocar 38 (shown in FIG. 2) generates position signals in response to sensing external magnetic fields generated by field generators 44, to enable a processor 34 to estimate the position of sensor 45 and thereby a position of a distal end of trocar 38.

This technique of position sensing is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, which prior applications are hereby incorporated by reference in their entirety herein into this application as if set forth in full.

In some embodiments, system 20 comprises a console 33, which comprises a memory 49, and a driver circuit 42 configured to drive field generators 44, via a cable 37, with suitable signals so as to generate magnetic fields in a predefined working volume in space around head 41.

Processor 34 is typically a general-purpose computer, with suitable front end and interface circuits for receiving images from camera 48 and for controlling other components of system 20.

In some embodiments, processor 34 is configured to register an image produced by camera 48 with a medical image, such as an MRI image. Processor 34 may further register the position of the distal end of the cannula that is estimated using position sensor 45. Processor 34 is configured to register the camera image and the reference medical image in the coordinate system of the magnetic position-tracking system and/or in a coordinate system of the reference medical image.

In some embodiments, system 20 comprises a video display 52 that shows an image 55 taken by camera 48. In the shown image, a distal end of treating probe 39 can be seen engaging brain tissue.

In some embodiments, processor 34 is configured to receive, via an interface (not shown), one or more anatomical images, such as reference MRI images depicting two-dimensional (2D) slices of head 41. Processor 34 is configured to select one or more slices from the MRI images, register it with a real-time camera image, such as image 55, to produce a combined image, such as an image 35, and display the selected combined slice to physician 24 on user display 36. In the example of FIG. 1, combined image 35 depicts a sectional coronal view of anterior brain tissue of patient 22.

Console 33 further comprises input devices, such as a keyboard and a mouse, for controlling the operation of the console, and a user display 36, which is configured to display the data (e.g., images) received from processor 34 and/or to display inputs inserted by a user using the input devices (e.g., by physician 24).

FIG. 1 shows only elements related to the disclosed techniques, for the sake of simplicity and clarity. System 20 typically comprises additional or alternative modules and elements that are not directly related to the disclosed techniques, and thus are intentionally omitted from FIG. 1 and from the corresponding description.

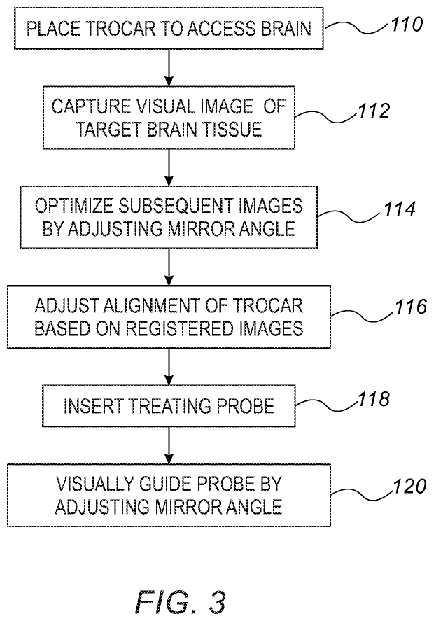

Processor 34 may be programmed in software to carry out the functions that are used by the system, and to store data in memory 49 to be processed or otherwise used by the software. The software may be downloaded to the processor in electronic form, over a network, for example, or it may be provided on non-transitory tangible media, such as optical, magnetic or electronic memory media. Alternatively, some or all of the functions of processor 34 may be carried out by dedicated or programmable digital hardware components. In particular, processor 34 runs a dedicated algorithm as disclosed herein, including in FIG. 3, that enables processor 34 to perform the disclosed steps, as further described below.

### Trocar with Internal Camera Providing Adjustable Angle of View

FIG. 2 is a schematic, pictorial illustration of the trocar 38 applied in the brain procedure of FIG. 1, in accordance with an embodiment of the present invention. As seen, trocar 38 includes a channel 70 inside a cannula 69, a camera 48, and a tiltable mirror 50 mounted on a distal edge of channel 70. Furthermore, channel 70 accommodates cable 32 for wiring the camera, the mirror and position sensor 45. The required wiring may be routed (61) to control handle 60, from which the wiring is routed to the console via a cable 32 of FIG. 1.

A zoom-in (100) on a distal end of cannula 69 shows that camera 48 is mounted with a distal viewing direction of a distal opening 78 of cannula 69. As seen, the viewing direction of camera 48 can be adjusted between a minimally angled (e.g., tilted) direction 82 and a maximally angled direction 84, covering a range θ angles of directions (e.g., 100 degrees), including a direction going to a center of the distal opening 78 of cannula 69.

Typically, to adjust camera viewing direction, the physician uses a knob (not shown) on external control handle 60. For example, a rotation of the knob sends a signal to tiltable mirror 50 (via camera cable 32) to change an angle of view. By rotating the knob in any direction, the physician can tilt mirror 50 to obtain a best image 55 on display 52.

In an embodiment, seen in inset 102, tiltable mirror 50 is a MEMS mirror that deflects a fixed viewing direction 51 of camera 48 according to a control voltage received by the mirror. However, other solutions may be used to adjust a view of camera 48, including other types of moving mirrors or optics, or by mounting camera 48 itself on a tiltable mount (and thereby avoiding a need for a tiltable mirror or other types of movable optics).

The configuration of trocar 38 in FIG. 2 is depicted by way of example for the sake of conceptual clarity. In other embodiments, additional elements may be included, such as additional ports in trocar 38, to insert medical tools, such as probe 39, to the target brain location.

FIG. 3 is a flow chart that schematically illustrates a method and algorithm for registering a visual image from camera 48 of trocar 38 of FIG. 2 with a reference medical image, in accordance with an embodiment of the present invention. The process begins when physician 24 places trocar 38 to access the brain, at a trocar placement step 110.

Next, in an image capturing step 112, physician 24 captures an image of target brain tissue by camera 48.

Typically, such a first image will not be the best possible view.

At an image adjustment step 114, physician 24 adjusts an angle of tiltable mirror 50 to capture better images of the target brain tissue. Next, at a trocar adjustment step 116, using the new angled views, physician 24 adjusts an alignment of trocar 38, e.g., to best allow best access to target brain tissue, such as infected tissue.

Physician 24 then inserts a treating probe 39, at a probe insertion step 118, to treat target tissue. During treatment, visual guidance (120) is provided by adjusting a view from camera 48 (by a adjusting a tilt angle of mirror 50), at a treatment guiding step 120.

The example flow chart shown in FIG. 3 is chosen purely for the sake of conceptual clarity. In alternative embodiments physician 24 may perform additional steps, such as employing additional monitoring steps (e.g., fluoroscopy) to verify the successful outcome of the procedure, and/or apply irrigation to clear a view for camera 48.

Although the embodiments described herein mainly address brain procedures, the methods and systems described herein can also be used, mutatis mutandis, in other applications that require guiding a medical device in other organs, such as located in the abdomen or the chest.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A trocar for insertion into an organ of a patient, the trocar comprising:
a cannula having a distal opening;
a channel inside the cannula; and
an optical assembly comprising a camera, which is disposed at a distal end of the channel and is configured to provide camera images of the distal opening with a field-of-view (FOV) that is tilted relative to a longitudinal axis of the cannula.

2. The trocar according to claim 1, wherein the optical assembly is configured to modify a tilt angle of the FOV in response to an adjustment by a user.

3. The trocar according to claim 2, wherein the optical assembly comprises a tiltable mirror that is configure to modify the tilt angle of the FOV by deflect a direction of view of the camera.

4. The trocar according to claim 3, wherein the tiltable mirror is a MEMS mirror.

5. The trocar according to claim 2, wherein the optical assembly comprises a tiltable element on which the camera is mounted so as to deflect a direction of view of the camera.

6. The trocar according to claim 5, wherein the tiltable element is a MEMS element.

7. A method, comprising:
inserting a trocar into an organ of a patient, the trocar comprising a cannula having a distal opening, and a channel inside the cannula; and
using an optical assembly comprising a camera that is disposed at a distal end of the channel, acquiring camera images of the distal opening with a field-of-view (FOV) that is tilted relative to a longitudinal axis of the cannula.

8. The method according to claim 7, and comprising modifying a tilt angle of the FOV in response to an adjustment by a user.

9. The method according to claim 8, wherein modifying the tilt angle comprises tilting a tiltable mirror that is configured to deflect a direction of view of the camera.

10. The method according to claim 9, wherein the tiltable mirror is a MEMS mirror.

11. The method according to claim 8, wherein modifying the tilt angle comprises tilting a tiltable element on which the camera is mounted so as to deflect a direction of view of the camera.

12. The method according to claim 11, wherein the tiltable element is a MEMS element.
